(19) United States
(12) Patent Application Publication
(10) Pub. No.: US 2009/0240291 A1
(43) Pub. Date: Sep. 24, 2009

(54) BREACHED PEDICLE SCREW

(75) Inventor: Josef Gorek, Ross, CA (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/410,315

(22) Filed: Mar. 24, 2009

Related U.S. Application Data
(60) Provisional application No. 61/070,533, filed on Mar. 24, 2008.

(51) Int. Cl. A61B 17/86

(52) U.S. Cl. 606/316; 606/301

(57) ABSTRACT

A bone screw apparatus adapted to replace a breached pedicle screw includes a proximal end and a distal end. The bone screw apparatus also includes an unthreaded eccentric portion and a threaded proximal portion and a threaded distal portion. The threaded proximal portion extends from the proximal end to the unthreaded eccentric portion. The threaded distal portion extends from the unthreaded eccentric portion to the distal end of the bone screw. In addition, the proximal portion and the distal portion each have a major diameter and a minor diameter.

### CROSS-REFERENCE TO RELATED APPLICATIONS

This application claims the benefit of, and priority to, U.S. Provisional Pat. Application Ser. No. 61/070,533 filed on Mar. 24, 2008, the entire contents of which are incorporated by reference herein.

### BACKGROUND

The present disclosure relates generally to an apparatus for stabilizing the joints of the spine during orthopedic spine surgery and more specifically, to a multi-planar bone screw for securing a spinal rod to a vertebra. Particularly, the present disclosure relates to a bone screw apparatus having an eccentric unthreaded portion to allow the bone screw to be placed even after a pedicle has been breached.

The spinal column is a complex system of bones and connective tissues that provide support for the human body and protection for the spinal cord and nerves. The adult spine is comprised of twenty-four vertebral bodies, which are subdivided into three areas, including seven cervical vertebrae, twelve thoracic vertebrae and five lumbar vertebrae. Between each vertebral body is an intervertebral disc that cushions and dampens the various translational and rotational forces exerted upon the spinal column.

There are various disorders, diseases, and types of injury which the spinal column may experience in a lifetime. These problems may include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function.

One of the more common solutions to any of the above-mentioned conditions involves a surgical procedure known as a spinal fusion. Spinal fusion involves fusing two or more vertebral bodies together to eliminate motion at the intervertebral disc or joint. To achieve spinal fusion, natural or artificial bone, along with a spacing device, replace part or the entire intervertebral disc to form a rigid column of bone. Mechanical hardware is connected to the adjacent vertebrae to stabilize the spine in that area while the bone grows and the fusion occurs.

The mechanical hardware used to immobilize the spinal column typically involves a series of bone screws and metal rods or plates. When the spine surgery is performed posteriorly, it is common practice to place pedicle bone screws into the vertebral bodies and then connect a metal rod between the screws, thus creating a rigid structure between adjacent vertebral bodies. In some cases, the use of these devices may be permanently implanted in the patient. In other cases, the devices may be implanted only as a temporary means of stabilizing or fixing the bones or bone fragments, with subsequent removal when no longer needed. It is also common that device implants that were intended to be permanent may require subsequent procedures or revisions as the dynamics of the subject's condition warrant. For these reasons, it is desirable that the implanted devices are easily implanted by the surgeon and are also configured to facilitate ease of removal, if required.

When using pedicle screws, the surgeon directs the screw through the pedicle into the vertebral body. Since different patients have different anatomies, there exists the potential for the pedicle to be breached. A pedicle is breached when the screw does not go directly through the pedicle channel and the screw protrudes through the bone on either the lateral or medial side. Often, if there is a lateral breach, the surgeon leaves the screw in place. If the breach occurs medially into the spinal canal, the spinal nerves can rub against the threads causing the patient pain and possibly requiring a revision surgery. Typically, when the surgeon recognizes the breach, he uses an instrument to displace the nerves to protect them from damage, removes the original screw and redirects it. Redirection removes more bone and can compromise fixation of the screw or completely damage the pedicle rendering it unusable as a point of device fixation.

To meet the problem of protecting the spinal nerves and preventing redirection, various types of bone fixation screws are available. One example is a device used for fixating fragments of bone together that is described in U.S. Pat. No. 5,019,079 issued to Ross. The proximal and distal portions have different thread pitches to allow for compression of the bone fragments together. Another design also used to fix bone fragments together is highlighted in U.S. Patent Application Publication No. 2005/0033300. In this design, the proximal and distal threads have the same pitch.

### SUMMARY

The present disclosure is directed towards an apparatus for stabilizing the joints of the spine during orthopedic spine surgery and more specifically, to a multi-planar bone screw for securing a spinal rod to a vertebra. In particular, embodiments of the present disclosure include a bone screw having an eccentric unthreaded portion to allow the bone screw to be placed even after the pedicle has been breached.

A bone screw apparatus adapted to replace a breached pedicle is disclosed herein. The bone screw includes an unthreaded eccentric middle portion and has a proximal end and a distal end. In one embodiment, the bone screw apparatus includes a threaded proximal portion that extends from the proximal end to the unthreaded eccentric portion and a threaded distal portion that extends from the unthreaded eccentric portion to the distal end of the bone screw apparatus. In addition, the proximal portion and the distal portion each have a major diameter and a minor diameter.

According to one aspect of the disclosure, the bone screw apparatus includes a curved unthreaded eccentric portion. In this embodiment, the curvature of the unthreaded portion may not extend past the major diameter of the proximal portion and may not extend past the major diameter of the distal portion.

In another embodiment, the proximal portion and the distal portion may have threads with the same pitch. In addition, it is envisioned that the proximal portion may have a different diameter than the distal portion. It is contemplated that the proximal portion may have a larger diameter than the distal portion or a smaller diameter than the distal portion.

In another aspect of the present disclosure, the bone screw apparatus may include a polyaxial housing. According to this embodiment, the housing is located at the proximal end of the bone screw apparatus. Moreover, the bone screw apparatus may also include a proximal housing that is configured to accept a spine fixation rod.

According to another aspect of the disclosure, the bone screw apparatus may include a reference mark to be used in determining the orientation of the eccentric portion.

### BRIEF DESCRIPTION OF THE DRAWINGS

Various embodiments of the presently disclosed breached pedicle screw are described hereinbelow with references to the drawings, wherein:

FIG. 1A is a top view of a first embodiment of a breached pedicle screw;

FIG. 1B is a front view of the breached pedicle screw of FIG. 1A;

FIG. 1C is an isometric view of the breached pedicle screw of FIG. 1A;

FIG. 2A is a side view of an additional embodiment of a breached pedicle screw with an eccentric unthreaded middle portion;

FIG. 2B is a side view of a further embodiment of a breached pedicle screw with an unthreaded middle portion;

FIG. 2C is a side view of another embodiment of a breached pedicle screw with an unthreaded top portion;

FIG. 2D is a top view of a head of a breached pedicle screw in accordance with the present disclosure;

FIG. 3A is a front view of a breached pedicle screw with a polyaxial head;

FIG. 3B is an isometric view of the breached pedicle screw of FIG. 3A;

FIG. 3C is an exploded view of the breached pedicle screw of FIG. 3A;

FIG. 4A is a front view of a housing:

FIG. 4B is a side view of the housing of FIG. 4A;

FIG. 5A is a top view of a screw coupling;

FIG. 5B is an isometric view of the screw coupling of FIG. 5A;

FIG. 5C is a side view of the screw coupling of FIG. 5A;

FIG. 6A is a side view of a screw insert; and

FIG. 6B is an isometric view of the screw insert of FIG. 6A.

### DETAILED DESCRIPTION OF THE EMBODIMENTS

Various embodiments of the presently disclosed bone screw apparatus will now be described in detail with reference to the drawings, wherein like reference numerals identify similar or identical elements. In the drawings and in the description that follows, the term “proximal,” will refer to the end of a device or system that is closest to the operator, while the term “distal” will refer to the end of the device or system that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front.

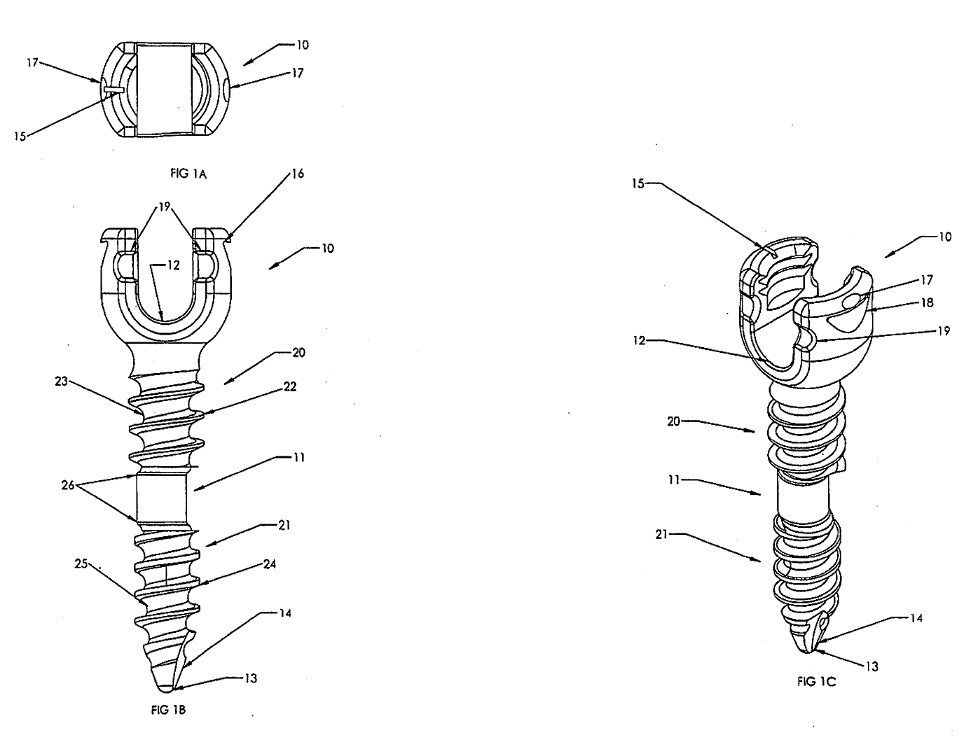

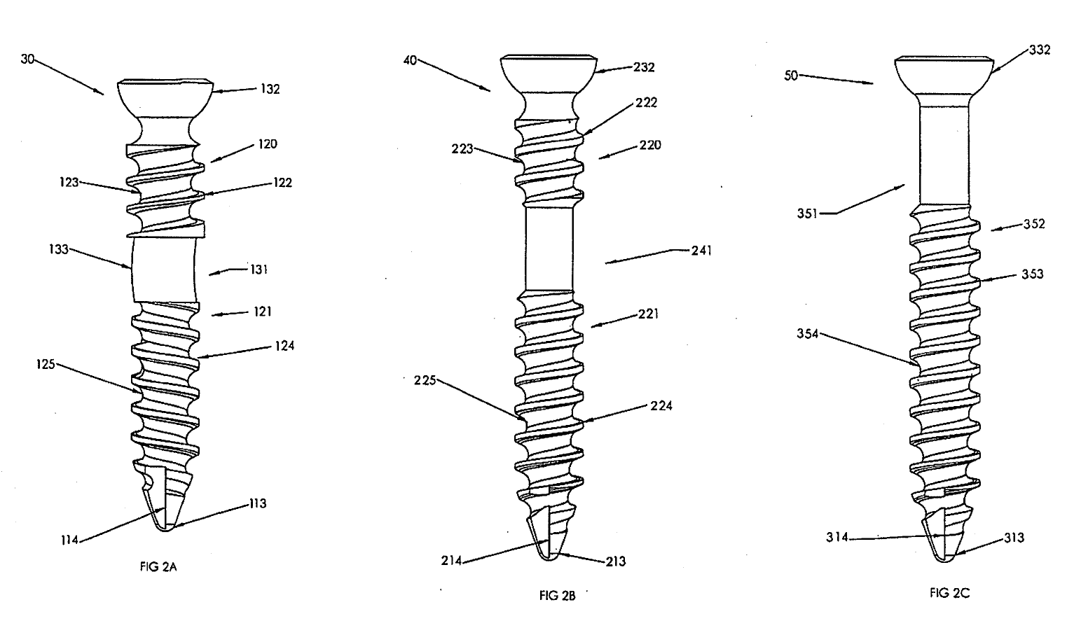

With reference to FIGS. 1A, 1B, and 1C, a breached pedicle screw or bone screw 10 is depicted with an eccentric unthreaded middle portion 11. The bone screw 10 further includes a proximal end and a distal end, where a distal tip 13 is located at the distal end of bone screw 10. FIG. 1A depicts is a top view of the proximal end of bone screw 10. A reference marker 15 is shown in FIG. 1A that can be used in determining the orientation of the eccentric unthreaded portion 11 with respect to the threaded portions of the bone screw 10.

Further, as shown best in FIGS. 1B and 1C, the bone screw 10 includes a proximal threaded portion 20 and a distal threaded portion 21. Proximal threaded portion 20 has a specific major diameter 22 and minor diameter 23. The distal threaded portion 21 has a specific major diameter 24 and minor diameter 25. At the distal tip 13 of the bone screw 10, there is a self-tapping feature 14. It is also contemplated that the distal tip 13 of the bone screw 10 may have a self-starting feature. At the proximal end of the bone screw 10, there is a saddle 12 which is configured and adapted for receiving a spine rod (not shown). Also at the proximal end, there is a lip 16, a semicircular groove 19, a keyway 18 and a lead in notch 17 which allow for the attachment of instruments used in surgery. Examples of these instruments can be found in U.S. patent application Ser. No. 12/104,653, filed on Apr. 17, 2008 and published as U.S. Patent Application Publication No. 2008/0262318. In this embodiment, the saddle 12 has a fixed orientation with respect to the threaded portions 20, 21 of the bone screw 10.

The proximal threaded portion 20 and the distal threaded portion 21 have the same pitch, but the major diameter 22 of the proximal threaded portion 20 is larger than the major diameter 24 of the distal threaded portion 21. The major diameter of the eccentric portion 11 is larger than the major diameter 24 of the distal threaded portion 21 but smaller than the major diameter 22 of the proximal threaded portion 20. The reference marker 15 allows the surgeon to know where a middle unthreaded section 26 is even after the device has been inserted into bone. In particular, the reference marker 15 identifies a portion of the unthreaded section 26 that extends further from a central longitudinal axis of the bone screw 10 than the portion that is 1800 away (FIG. 1B).

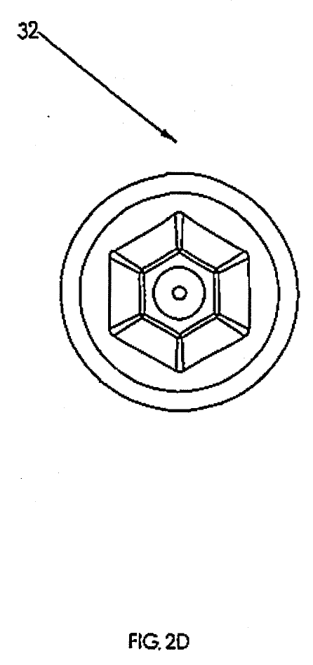

With reference to FIGS. 2A, 2B, and 2C, low profile embodiments of bone screws 30, 40 and 50 are shown, while FIG. 2D shows a top view 32 of bone screws 30, 40, and 50. FIG. 2A depicts a curved bone screw 30 which includes a low profile head 132 that has a frusto-conical feature that allows for driving the bone screw 30 into vertebrae or other bones. Bone screw 30 further includes a middle curved unthreaded portion 131, a proximal threaded portion 120 with a specific major diameter 122 and a minor diameter 123, a distal threaded portion 121 with a specific major diameter 124 and a minor diameter 125, and a distal tip 113 with self-tapping feature 114. The curved unthreaded portion 131 has a curvature 133. The major diameter 122 of the proximal threaded portion 120 is larger than the major diameter 124 of the distal threaded portion 121. The curvature 133 of the middle portion 131 does not extend past the major diameter 122 of the proximal threaded portion 120.

FIG. 2B shows a bone screw 40 that includes a low profile head 232, a middle unthreaded portion 241, a proximal threaded portion 220 with a specific major 222 and a minor 223 diameters, a distal threaded portion 221 with a specific major diameter 224 and a minor diameter 225, and a distal tip 213 with a self-tapping feature 214. The low profile head 232 has a frusto-conical feature that allows for driving the screw 40 into vertebrae. The major diameter 222 and minor diameter 223 of the proximal threaded portion 220 are equal to the major diameter 224 and minor diameter 225 of the distal threaded portion 221. The middle unthreaded portion 241 has the same diameter as the minor diameter 225 of the distal threaded portion 221.

FIG. 2C shows a bone screw 50 that includes a low profile head 332, a proximal unthreaded portion 351 and a distal threaded portion 352. Distal threaded portion 352 further includes distal threads with a specific major diameter 353 and a minor diameter 354, and a distal tip 313 with a self-tapping feature 314. The low profile head 332 has a frusto-conical feature that allows for driving bone screw 50 into vertebral bone. The proximal unthreaded portion 351 is on the same axis as the rest of the bone screw 50. The proximal unthreaded portion 351 also has the same diameter as the minor diameter 354 of the distal threaded portion 352.

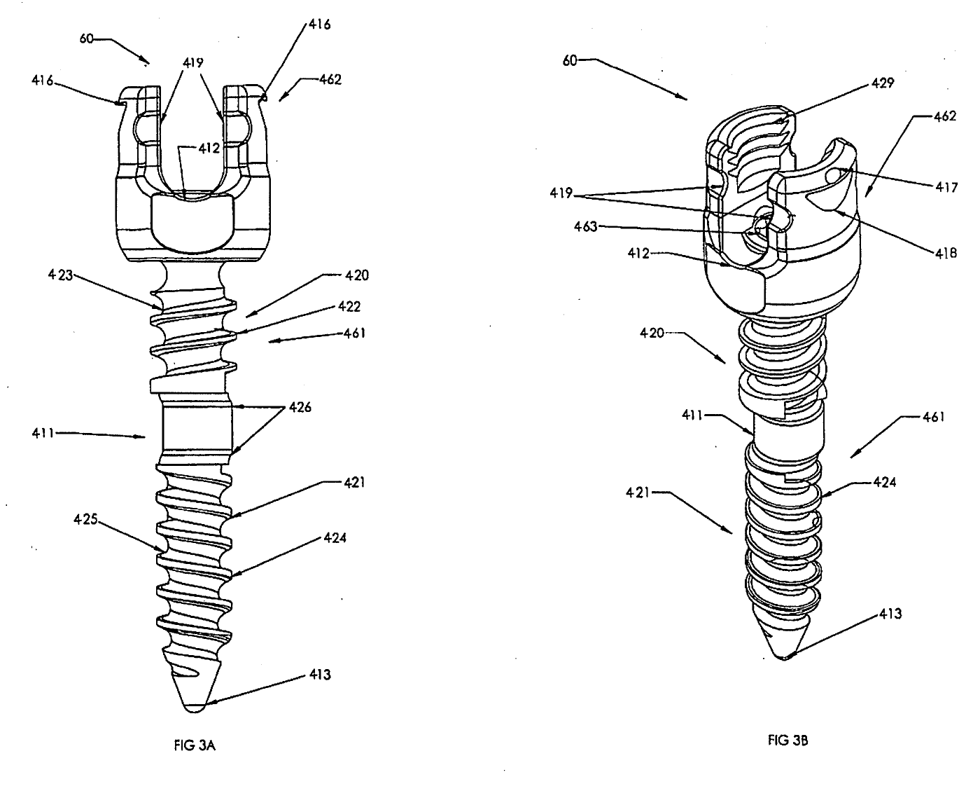

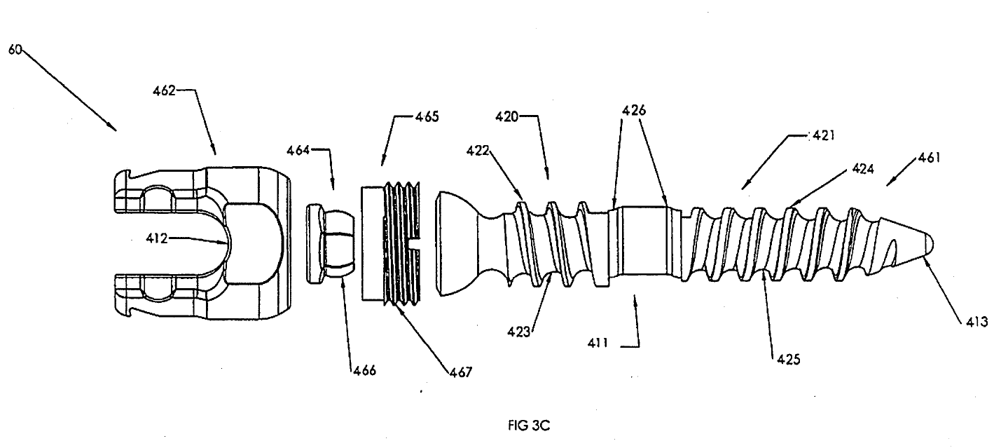

With reference to FIGS. 3A, 3B, and 3C, a polyaxial embodiment of a bone screw 60 is depicted. Bone screw 60 includes a proximal threaded portion 420 with a specific major diameter 422 and a minor diameter 423, a distal threaded portion 421 with a specific major diameter 424 and a minor diameter 425, and a distal tip 413 with a self-tapping feature 414. The bone screw 60 can be replaced by any of the low profile embodiments of bone screws 30, 40, or 50 disclosed hereinabove. Further still, the bone screw 60 includes an unthreaded eccentric portion 411 similar to that previously discussed with respect to bone screws 10, 30, 40, and 50.

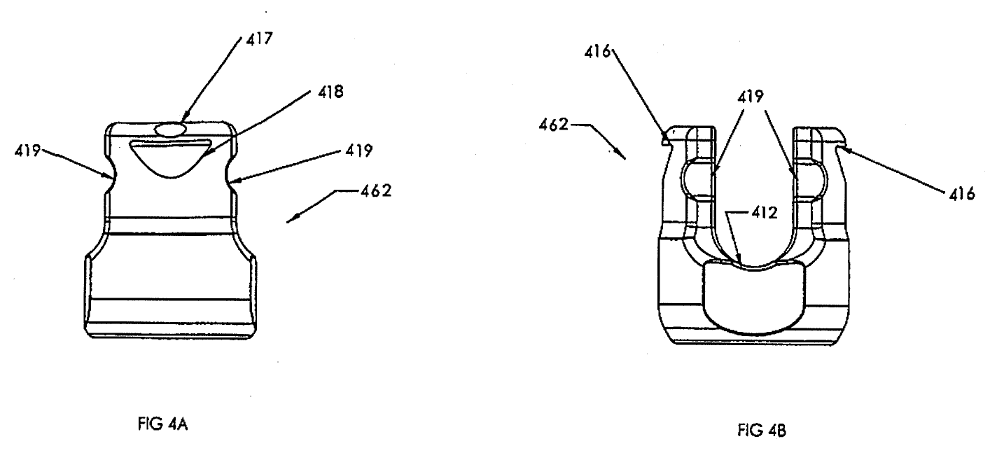

The polyaxial bone screw 60 further includes a polyaxial housing 462, a screw-coupling 464, and a threaded screw insert 465 with external threads 467. The polyaxial housing 462 of bone screw 60 is depicted separate from bone screw 60 in FIGS. 4A and 4B. As shown in FIGS. 3A, 3B, 4A, and 4B, polyaxial housing 462 has a saddle 412 and an internal threaded portion 429 used to lock a rigid member such as a rod or plate (not shown) into the saddle 412. Polyaxial housing 462 may also include a lip 416, a semicircular groove 419, a keyway 418 and a lead in notch 417 which would allow for the attachment of instruments used in surgery. Examples of these instruments can be found in U.S. patent application Ser. No. 12/104,653, filed on Apr. 17, 2008 and published as U.S. Patent Application Publication No. 2008/0262318, the entire contents of which are incorporated by reference herein.

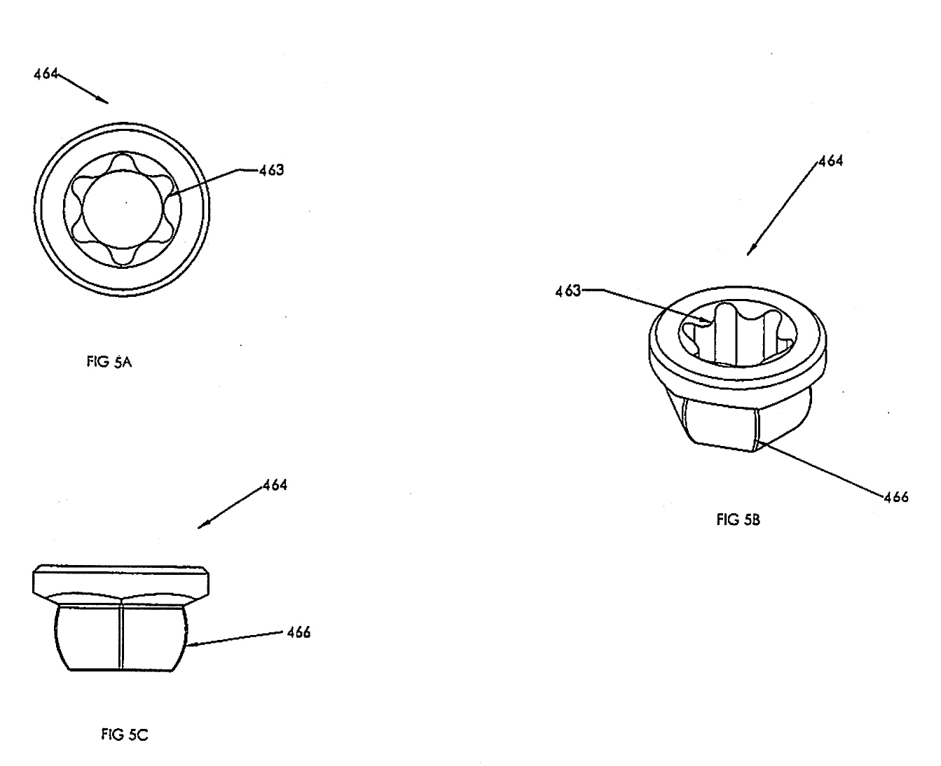

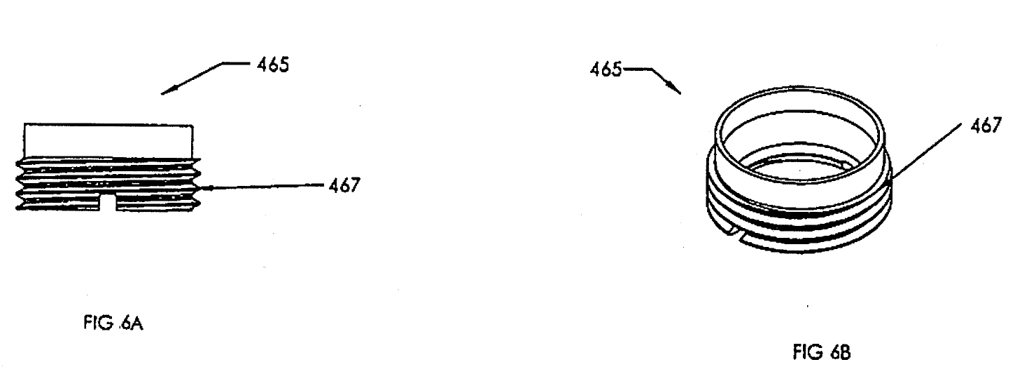

The screw-coupling device 464 is depicted separate from bone screw 60 in FIGS. 5A, 5B, and 5C. As shown first in FIG. 3C, screw-coupling device 464 has a frusto-conical distal end 466 that mates with a frusto-conical feature of the bone screw 60. As shown in FIGS. 5A, 5B, and 5C, the screw-coupling device 464 also has a hexalobular cut out 463 that allows an instrument to drive the bone screw 60 into vertebrae.

The screw insert 465 is depicted separate from the bone screw 60 in FIGS. 6A and 6B. As shown best in FIG. 3C, screw insert 465 locks the screw-coupling device 464 into the screw head 462 by mating the internal threads 429 of the screw head 462 and the external threads 467 of the screw insert 465.

In use, the bone screw 60 is inserted through the housing 462. The inner portion of the housing 462 is configured and adapted to seat the head of the bone screw 60 while allowing the threaded portions 420, 421 and the unthreaded portion 411 of the bone screw 60 to extend distally beyond the housing 462. As such, the bone screw 60 is rotatable and pivotable with respect to the housing 462. The screw insert 465 is inserted over the threads of the bone screw 60 into the base of the housing 462 where the external threading of the screw insert 465 mates with internal thread of the housing 462. Tightening the screw insert 465 with respect to the housing 462 secures the bone screw 60 in the housing 462 while permitting polyaxial movement of the bone screw 60 with respect to the housing 462. The coupling 464 may be inserted into the head of the bone screw 60 where outer surfaces of the frusto-conical distal end 466 engage the surfaces of the recess in the bone screw 60. This arrangement allows a driving tool to be used that may be off-axis from the central axis of the bone screw 60 while permitting the bone screw 60 to be driven into bone. In embodiments where the recess in the head of the bone screw 60 is not hexagonal, the coupling 464 has a complementary distal end suitable for engaging the recess. Other suitable bone screws may be used such as those disclosed in International Patent Application No. PCT/US2008/080668, filed on Oct. 22, 2008, the entire contents of which are hereby incorporated by reference.

During a procedure, if the pedicle is breached using a conventional bone screw, the physician removes the conventional bone screw and inserts the presently disclosed breached pedicle screw 10. Depending on the geometry of the breach, the physician may select one of the other disclosed embodiments of the breached pedicle screw 60. Since the outer thread diameter of the breached pedicle screw 10, 60 is greater than that of the conventional bone screw, the breached pedicle screw 10, 60 provides improved purchase when inserted into bone. The breached pedicle screw 10, 60 is inserted to a predetermined depth and the reference mark 15 indicates the orientation of the eccentric unthreaded portion of the respective breached pedicle screw 10, 60. By using the reference mark 15 to adjust the position of the unthreaded eccentric portion, the physician orients the breached pedicle screw 10, 60 such that there is additional space for the nerve roots, thereby minimizing trauma to the patient. As such, the breached pedicle screw 10, 60 provides sufficient anchoring capabilities while still accepting a fixation device.

The bone screw disclosed hereinabove may be modified according to the present disclosure. For example, the bone screw may be cannulated. In addition, the bone screw may be have a number of different lengths and diameters. Moreover, the apparatus used in driving the bone screw into the vertebrae may be something other than a hex or square drive, such as Phillips or hexalobular.

It should be understood that the present disclosure is not limited to the precise embodiments discussed herein above, and that various other changes and modifications may be contemplated by one skilled in the art without departing from the scope or spirit of the present disclosure. For example, the bone screw described herein above may be formed from a variety of surgically acceptable materials including titanium, plastics, bio-absorbable materials, etc. Although the illustrative embodiments of the present disclosure have been described herein with reference to the accompanying drawings, the above description, disclosure, and figures should not be construed as limiting, but merely as exemplary of various embodiments.

What is claimed is:

1. A bone screw apparatus adapted to replace a breached pedicle screw comprising:
a proximal end;
a distal end;
an unthreaded eccentric portion; and
a threaded proximal portion extending from the proximal end to the unthreaded eccentric portion and a threaded distal portion extending from the unthreaded eccentric portion to the distal end of the bone screw, the proximal portion and the distal portion each having a major diameter and a minor diameter.

2. The bone screw apparatus according to claim 1, wherein the unthreaded eccentric portion is curved.

3. The bone screw apparatus according to claim 2, wherein the curvature of the unthreaded eccentric portion does not extend past the major diameter of the proximal portion and does not extend past the major diameter of the distal portion.

4. The bone screw apparatus according to claim 1, wherein the proximal portion and the distal portion have threads with the same pitch.

5. The bone screw apparatus according to claim 1, wherein the proximal portion has a different diameter than the distal portion.

6. The bone screw apparatus according to claim 1, wherein the proximal portion has a larger diameter than the distal portion.

7. The bone screw apparatus according to claim 1, wherein the bone screw includes a polyaxial head portion.

8. The bone screw apparatus according to claim 1, wherein the bone screw includes a proximal head portion configured to accept a spine fixation rod.

9. The bone screw apparatus according to claim 1, further including a reference mark that corresponds to a portion of the eccentric portion.
